(19) United States
(12) Patent Application Publication
(10) Pub. No.: US 2008/0003600 A1
(43) Pub. Date: Jan. 3, 2008

(54) ISOTHERMAL SCREENING OF BREAST CANCER RELATED NUCLEIC ACID

(76) Inventor: Frank G. Salinas, Wheaton, IL (US)

Correspondence Address: MCANDREWS HELD & MALLOY, LTD, 500 WEST MADISON STREET, SUITE 3400, CHICAGO, IL, 60661, US

(21) Appl. No.: 11/679,709

(22) Filed: Feb. 27, 2007

Related U.S. Application Data
(60) Provisional application No. 60/777,022, filed on Feb. 27, 2006.

(51) Int. Cl. C12Q 1/68

(52) U.S. Cl. 435/6

(57) ABSTRACT

The presently described technology relates generally to the art of molecular diagnostics and more particularly to point-of-care diagnostic methods and materials. The diagnostic methods and materials of the presently described technology are suitable for a variety of uses including but not limited to the bedside or field diagnosis of infectious or noninfectious diseases.

### RELATED APPLICATIONS

The present application is related to and claims priority from U.S. Provisional Patent Application Ser. No. 60/777,022, filed Feb. 27, 2006, the contents of which are hereby incorporated herein by reference in their entirety. Additionally, all cited references in the present application are hereby incorporated by reference in their entirety.

### FEDERALLY SPONSORED RESEARCH OR DEVELOPMENT

[Not Applicable]

### MICROFICHE/COPYRIGHT REFERENCE

[Not Applicable]

### BACKGROUND OF THE INVENTION

The presently described technology relates generally to the art of molecular diagnostics and more particularly to point-of-care diagnostic methods and materials. The diagnostic methods and materials of the presently described technology are suitable for a variety of uses including but not limited to the diagnosis of cancer. In particular, the present invention relates to methods and materials for detecting breast cancer.

Breast cancer is the most common form of cancer occurring in females in the U.S. The incidence of breast cancers in the United States is projected to be 180,300 cases diagnosed and 43,900 breast cancer-related deaths to occur during 1998 (American Cancer Society statistics). Worldwide, the incidence of breast cancer increased from 700,000 in 1985 to about 900,000 in 1990. G. N. Hortobagyi et al., CA Cancer J Clin 45:199-226 (1995).

Procedures used for detecting, diagnosing, staging, monitoring, prognosticating, in vivo imaging, preventing or treating, or determining predisposition to diseases or conditions of the breast, such as breast cancer, are of critical importance to the outcome of the patient. For example, patients diagnosed with early breast cancer have greater than a 90% five-year relative survival rate as compared to a survival rate of about 20% for patients diagnosed with distantly metastasized breast cancers. (American Cancer Society statistics). Currently, the best initial indicators of early breast cancer are physical examination of the breast and mammography. J. R. Harris et al. In: Cancer: Principles and Practice of Oncology, Fourth Edition, pp. 1264-1332, Philadelphia, Pa.: J/B. Lippincott Co. (1993). Mammography may detect a breast tumor before it can be detected by physical examination, but it has limitations. For example, mammography's predictive value depends on the observer's skill and the quality of the mammogram. In addition, 80 to 93% of suspicious mammograms are false positives, and 10 to 15% of women with breast cancer have false negative mammograms. C. J. Wright et al., Lancet 346:29-32 (1995). New diagnostic methods that are more sensitive and specific for detecting early breast cancer are clearly needed.

Breast cancer patients are closely monitored following initial therapy and during adjuvant therapy to determine response to therapy, and to detect persistent or recurrent disease, or early distant metastasis. Current diagnostic procedures for monitoring breast cancer include mammography, bone scan, chest radiographs, liver function tests and tests for serum markers. The serum tumor markers most commonly used for monitoring patients are carcinoembryonic antigen (CEA) and CA 15-3. Limitations of CEA include absence of elevated serum levels in about 40% of women with metastatic disease. In addition, CEA elevation during adjuvant therapy may not be related to recurrence but to other factors that are not clinically important. CA 15-3 can also be negative in a significant number of patients with progressive disease and, therefore, fail to predict metastasis. Both CEA and CA 15-3 can be elevated in nonmalignant, benign conditions giving rise to false positive results. Therefore, it would be clinically beneficial to find a breast associated marker which is more sensitive and specific in detecting cancer recurrence. J. R. Harris et al., supra. M. K. Schwartz, In: Cancer: Principles and Practice of Oncology. Vol. 1, Fourth Edition, pp. 531-542, Philadelphia, Pa.: J/B. Lippincott Co. 1993.

Another important step in managing breast cancer is to determine the stage of the patient's disease because stage determination has potential prognostic value and provides criteria for designing optimal therapy. Currently, pathological staging of breast cancer is preferable over clinical staging because the former gives a more accurate prognosis. J. R. Harris et al., supra. On the other hand, clinical staging would be preferred were it at least as accurate as pathological staging because it does not depend on an invasive procedure to obtain tissue for pathological evaluation. Staging of breast cancer could be improved by detecting new markers in serum or urine which could differentiate between different stages of invasion. Such markers could be mRNA or protein markers expressed by cells originating from the primary tumor in the breast but residing in blood, bone marrow or lymph nodes and could serve as sensitive indicators for metastasis to these distal organs. For example, specific protein antigens and mRNA, associated with breast epithelial cells, have been detected by immunohistochemical techniques and RT-PCR, respectively, in bone marrow, lymph nodes and blood of breast cancer patients suggesting metastasis. K. Pantel et al., Onkologie 18:394-401 (1995).

Such diagnostic procedures also could include immunological assays based upon the appearance of various disease markers in test samples such as blood, plasma, serum or urine obtained by minimally invasive procedures which are detectable by immunological methods. These diagnostic procedures would provide information to aid the physician in managing the patient with disease of the breast, at low cost to the patient. Markers such as prostate specific antigen (PSA) and human chorionic gonadotropin (hCG) exist and are used clinically for screening patients for prostate cancer and testicular cancer, respectively. For example, PSA normally is secreted by the prostate at high levels into the seminal fluid, but is present in very low levels in the blood of men with normal prostates. Elevated levels of PSA protein in serum are used in the early detection of prostate cancer or disease in asymptomatic men. See, for example, G. E. Hanks et al., In: Cancer: Principles and Practice of Oncology, Vol. 1, Fourth Edition, pp. 1073-1113, Philadelphia, Pa.: J. B. Lippincott Co. 1993. M. K. Schwartz et al., In: Cancer: Principles and Practice of Oncology, Vol. 1, Fourth Edition, pp. 531-542, Philadelphia, Pa.: J. B. Lippincott Co. 1993. Likewise, the management of breast diseases could be improved by the use of new markers normally expressed in the breast but found in elevated amounts in an inappropriate body compartment as a result of the disease of the breast.

Further, new markers which could predict the biologic behavior of early breast cancers would also be of significant value. Early breast cancers that threaten or will threaten the life of the patient are more clinically important than those that do not or will not be a threat. G. E. Hanks, supra. Such markers are needed to predict which patients with histologically negative lymph nodes will experience recurrence of cancer and also to predict which cases of ductal carcinoma in situ will develop into invasive breast carcinoma. More accurate prognostic markers would allow the clinician to accurately identify early cancers localized to the breast which will progress and metastasize if not treated aggressively. Additionally, the absence of a marker for an aggressive cancer in the patient could spare the patient expensive and non-beneficial treatment. J. R. Harris et al., supra. E. R. Frykberg et al., Cancer 74:350-361 (1994).

One main challenge is to intensify the develop of early detection platforms. There is a need for an easy, home or point-of-care molecular diagnostic system for the amplification and detection of nucleic acids related to the development or onset of breast cancer.

### BRIEF SUMMARY OF THE INVENTION

One object of the present invention is to provide a molecular diagnostic system comprising methods and materials for the isothermal detection and screening of nucleic acids. Still another object of the present invention is to provide a molecular diagnostic system comprising methods and reagents for the isothermal detection and screening of nucleic acids associated with but not limited to disease, disease predisposition, disease causative agents, and any combination or derivative thereof. A further object of the present invention is to provide a molecular diagnostic system comprising methods and materials for the isothermal detection and screening of nucleic acids associated with cancer.

One or more of the preceding objects, or one or more other objects which will become plain upon consideration of the present specification, are satisfied by the invention described herein.

One aspect of the invention, which satisfies one or more of the above objects, is a test kit having reagents for the isothermal detection of nucleic acids associated with but not limited to disease, disease predisposition, disease causative agents, and any combination or derivative thereof. Another aspect of the invention is a test kit comprising: a strand transferase component; a polymerase component; and one or more primers and/or probes complementary to one or more nucleic acids associated with but not limited to disease, disease predisposition, disease causative agents, and any combination or derivative thereof. One preferred aspect of the present invention is a test kit comprising: a reverse transcriptase, a strand transferase component; a DNA dependent DNA polymerase component; and one or more primers and/or probes complementary to one or more nucleic acids associated with breast cancer.

### BRIEF DESCRIPTION OF SEVERAL VIEWS OF THE FIGURES

FIG. 1 is a schematic view of one aspect of the isothermal DNA amplification system of the present invention employing one primer complementary to a target nucleic acid, a strand transferase, and a polymerase.

FIG. 2 is a schematic view of another aspect of the isothermal DNA amplification system of the present invention employing two primers complementary to opposite strands and flanking a target nucleic acid, a strand transferase and a polymerase.

### DETAILED DESCRIPTION OF THE INVENTION

The present invention provides methods and materials for the isothermal screening and detection of nucleic acids associated with but not limited to disease, disease predisposition, disease causative agents, and any combination or derivative thereof. As used herein, and without limitation, nucleic acid generally includes any size DNA, RNA, DNA/RNA hybrid, or analog thereof. The nucleic acid can be single stranded, double stranded, or a combination of single and double stranded. As used herein, and without limitation, disease generally includes an impairment of the normal state of the living animal or plant body or one of its parts that interrupts or modifies the performance of the vital functions, is typically manifested by distinguishing signs and symptoms, and is a response to environmental factors (as malnutrition, industrial hazards, or climate), to specific infective agents (as parasites, bacteria, or viruses), to inherent defects of the organism (as genetic anomalies), or to combinations or derivatives of these factors.

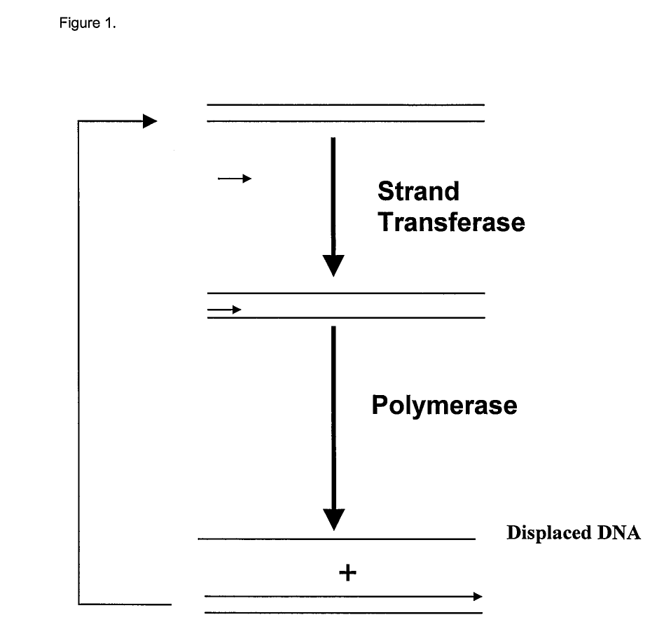

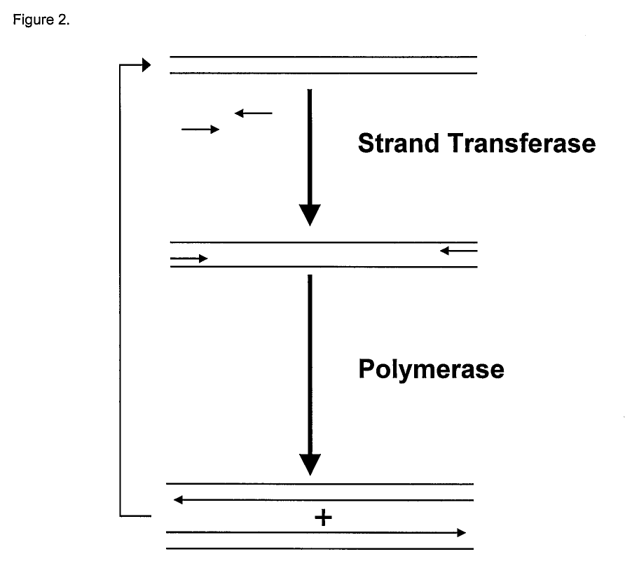

One aspect of the present invention includes methods and materials for the quantitative or qualitative isothermal screening and detection of one or more target nucleic acids of interest. This aspect of the present invention comprises contacting the target nucleic acid with at least one nucleic acid primer having complementarity to the target nucleic acid, a strand transferase, and a polymerase. The strand transferase catalyzes the homologous pairing of the at least one primer to a specific location on the target nucleic acid to form a primer-template junction that is acted upon by the polymerase to replicate and amplify the target nucleic acid (FIG. 1). In one preferred embodiment, the target nucleic acid is contacted with two primers complementary to opposite strands and flanking said target nucleic acid, in the presence of a strand transferase and a polymerase (FIG. 2). In certain aspects of the present invention, the isothermal amplification of the nucleic acid is performed as describe in U.S. Pat. No. 6,929,915, Methods for Nucleic Acid Manipulation. This reference is herein incorporated by reference.

As used herein without limitation, a strand transferase generally is a catalyst for the identification and base pairing of homologous sequences between nucleic acids, a process also known as homologous pairing or strand exchange. Bianco et al provides a general discussion of strand transferases in “DNA strand exchange proteins: a biochemical and physical comparison” at Front Biosci. 1998 Jun. 17; 3:D570-603. This reference is herein incorporated by reference. Strand transferases can be derived from either a prokaryotic system or an eukaryotic system, including but not limited to yeast, bacteria, and bacteriophages such as T4 and T7. For example West discusses eukaryotic strand transferases in Recombination genes and proteins” in Curr Opin Genet Dev. 1994April;4(2):221-8. This reference is herein incorporated by reference. Radding discussed the recA strand exchange protein in “Helical RecA nucleoprotein filaments mediate homologous pairing and strand exchange” at Biochim Biophys Acta. 1989 Jul. 7, 1008(2):131-45. This reference is herein incorporated by reference. Also, the UvsX strand transferase was described by Kodadek et al. The mechanism of homologous DNA strand exchange catalyzed by the bacteriophage T4 uvsX and gene 32 proteins” JBC 1989 Jul. 5; 263(19):9427-36. This reference is herein incorporated by reference. Yonesaki discusses T4 homologous recombination in “Recombination apparatus of T4 phage” at Adv Biophys. 1995; 31:3-22. This reference is herein incorporated by reference. Also, Salinas et. al have discussed the homology dependence of UvsX catalyzed strand exchange in “Homology dependence of UvsX protein-catalyzed joint molecule formation” at J Biol Chem. 1995 Mar. 10, 1995;270(10):5181-6. This reference is herein incorporated by reference. Exemplar strand transferase proteins include but are not limited to the eukaryotic Rad51 protein, the bacterial recA protein, the bacterial phage T4 UvsX protein, the bacteriophage T7 gene 2.5 or any protein fragment, derivative, or homolog thereof, including proteins found in nature and those engineered or modified using recombinant DNA technology. Kong et. al has discussed T7 strand exchange in “Role of the bacteriophage T7 and T4 single-stranded DNA-binding proteins in the formation of joint molecules and DNA helicase-catalyzed polar branch migration.” J Biol Chem. 1997 Mar. 28; 272(13):8380-7. This reference is herein incorporated by reference.

Strand transferases generally operate by first binding single stranded regions of DNA to form a nucleoprotein filament generally referred to as the presynaptic filament. The presynaptic filament then binds a target nucleic acid and performs a search for homology that once complete results in the formation of a joint molecule or D-loop. Strand transferases generally have accessory protein factors that augment or modify their activity. For example, strand transferases generally have accessory protein factors that effect the formation and/or stability of the presynaptic filament under varying conditions, including for example buffer conditions and/or the presence of other proteins competing to bind regions of single-stranded nucleic acid. Exemplar strand transferase accessory proteins include but are not limited to the bacteriophage T4 UvsX accessory protein UvsY, the E. coli RecA accessory proteins RecFOR, the yeast and human Rad51 accessory protein Rad52, and any protein fragment, derivative, or homolog thereof, including proteins found in nature and those engineered or modified using recombinant DNA technology.

As used herein without limitation, a polymerase generally is any of several enzymes, such as DNA polymerase, RNA polymerase, or reverse transcriptase, that catalyze the formation of nucleic acid from precursor substances in the presence of preexisting nucleic acid acting as a template. The polymerase of the present invention can be derived from a eukaryotic or a prokaryotic system. For example the polymerase can be derived from a bacterium such as E. coli, a bacteriophage such as bacteriophage T4 or bacteriophage T7, a eukaryotic organism such as yeast or human, a virus, or any protein fragment, derivative, or homolog thereof, including proteins found in nature and those engineered or modified using recombinant DNA technology. Exemplar polymerases include but are not limited to the bacteriophage T4 gene product 43 protein, and any mutants or derivatives of the gene 43 protein including but not limited to the exonuclease deficient 43 exo⁻ polymerase. Benkovic et. al discusses replisome mediated DNA replication in “Replisome Mediated DNA Replication” at Annu Rev Biochem. 2001; 70:181-208. This reference is herein incorporated by reference.

Polymerases generally have accessory protein factors that augment or modify their activity. Exemplar polymerase accessory factors include but are not limited to clamp proteins and clamp loader proteins. Clamp proteins generally have affinity and/or a topological link to both the polymerase and the nucleic acid being acted upon by said polymerase, thereby forming a stable link between polymerase and nucleic acid, the result of which is the formation of a stable polymerase nucleic acid complex having high processivity Clamp loader proteins facilitate the assembly of a clamp protein onto a nucleic acid and can also facilitate and mediate a concomitant or subsequent interaction with the polymerase. As used herein in connection with certain aspects and embodiments of the invention, the term holoenzyme generally regards a polymerase-clamp complex.

Polymerase accessory factors can be derived from a bacterium such as E. coli, a bacteriophage such as bacteriophage T4 or bacteriophage T7, a eukaryotic organism such as yeast or human, a virus, or any protein fragment, derivative, or homolog thereof, including proteins found in nature and those engineered or modified using recombinant DNA technology. Exemplar clamp proteins include but are not limited to the bacteriophage T4 gene product 45 protein, and any mutants or derivatives of the T4 gene product 45 protein. Trakselis et discuss the T4 polymerase holoenzyme in Creating a dynamic picture of the sliding clamp during T4 DNA polymerase holoenzyme assembly by using fluorescence resonance energy transfer” at Proc Natl Acad Sci 2001 USA. Jul. 17; 98(15):8368-75. This reference is herein incorporated by reference.

In certain embodiments of the present invention, the quantitative or qualitative isothermal screening and detection of one or more target nucleic acids of interest is performed in the presence of a single stranded nucleic acid binding protein (SSB). SSB's used pursuant to the present invention can be derived from a bacterium such as E. coli, a bacteriophage such as bacteriophage T4 or bacteriophage T7, a eukaryotic organism such as yeast or human, or any protein fragment, derivative, or homolog thereof, including proteins found in nature and those engineered or modified using recombinant DNA technology. Exemplar SSB's include but are not limited to the E. coli SSB protein, the bacteriophage T4 gene product 32 protein, the bacteriophage T7 gene product 2.5 protein, and the yeast or human RPA protein, or any mutants or derivatives thereof.

In certain embodiments of the present invention, the quantitative or qualitative isothermal screening and detection of one or more target nucleic acids of interest is performed in the presence of a helicase, preferably a DNA helicase. The helicase can be derived from a prokaryote or a eukaryote. For example, the DNA helicase can be from a bacterium such as E. coli, a bacteriophage such as bacteriophage T4 or bacteriophage T7, a yeast, or human. Exemplar helicases include but are not limited to the bacteriophage T4 gene product 41, the bacteriophage T4 dda protein, the bacteriophage T7 gene 4 protein, the E.coli UvrD protein, and any mutants or derivatives thereof. For example, Salinas and Kodadek have discussed the role of DNA helicases during strand homologous recombination in “Phage T4 homologous strand exchange: a DNA helicase, not the strand transferase, drives polar branch migration.” Cell 1995 Jul. 14; 82(1):111-9. This reference is herein incorporated by reference. Also, Salinas and Benkovic have discussed the role of DNA helicases in bacteriophage T4 replication in “Characterization of bacteriophage T4-coordinated leading- and lagging-strand synthesis on a minicircle substrate.” Proc Natl Acad Sci USA. 2000 Jun. 20; 97(13):7196-201. This reference is herein incorporated by reference. Also, Alberts et al discusses the general nature of replication in bacteriophage T4 in “Studies on DNA replication in the bacteriophage T4 in vitro system” at Cold Spring Harb Symp Quant Biol. 1983; 47 Pt 2:655-68. This reference is herein incorporated by reference.

In certain other embodiments of the present invention, the quantitative or qualitative isothermal screening and detection of one or more target nucleic acids of interest is performed in the presence of a helicase and a helicase accessory factor. The DNA helicase and the DNA helicase accessory factor can be derived from a eukaryotic or prokaryotic system. For example, the DNA helicase and the DNA helicase accessory factor can be from a bacterial system such as E. coli. or a bacteriophage system such as bacteriophage T4. For example, one DNA helicase/accessory factor pair is the bacteriophage T4 gene product 41 protein and its accessory factor gene product 59 protein. Jones et al discusses the gene product 59 protein in “Bacteriophage T4 gene 41 helicase and gene 59 helicase-loading protein: a versatile couple with roles in replication and recombination” at Proc Natl Acad Sci USA. 2001 Jul. 17; 98(15):8312-8. This reference is herein incorporated by reference.

In still other embodiments of the present invention, the quantitative or qualitative isothermal screening and detection of one or more target nucleic acids of interest is performed in the presence of a primosome. As used herein a primosome is a term that generally characterizes a complex comprising a DNA helicase and an RNA polymerase usually referred to as a primase. The primosome is active in synthesizing RNA primers on the lagging strand of a replication fork for the initiation of Okazaki fragment synthesis during coordinated leading- and lagging strand synthesis. Primases can be derived from a prokaryote or a eukaryote. For example, the primase can be from a bacterium such as E. coli, a bacteriophage such as bacteriophage T4 or bacteriophage T7, a yeast, or a human. One exemplar primase is the bacteriophage T4 gene product 61 protein, and derivatives or mutants thereof.

The phrase “amplification reaction reagents” as used herein includes but is not limited to reagents which are well known for their use in nucleic acid amplification reactions and may include but are not limited to: a single or multiple reagent, reagents, enzyme or enzymes separately or individually having reverse transcriptase and/or polymerase activity, strand transferase activity, or exonuclease activity; enzyme cofactors such as magnesium or manganese; salts; nicotinamide adenine dinucleotide (NAD); and deoxynucleoside triphosphates (dNTPs) such as, for example, deoxyadenosine triphosphate, deoxyguanosine triphosphate, deoxycytodine triphosphate and thymidine triphosphate. Other reagents include molecular crowding agents, including but not limited to polyethylene glycol PEG 8000. The exact amplification reagents employed are largely a matter of choice for one skilled in the art based upon the particular amplification reaction employed. For example, it is known in the art that volume occuping agents, or molecular crowding agents, inhance the activity or function of strand transferases, polymerases, and their accessory factors. The following references are herein incorporated by reference: (1) “Enhancement of recA Protein-promoted DNA Strand Exchange Activity by Volume occupying agents” at J Biol Chem. 1992 May 5 267(13):9307-14; (2) “Stimulation of the processivity of the DNA polymerase of bacteriophage T4 by the polymerase accessory proteins” at J Biol Chem. 1991 Jan. 25; 266(3):1830-40; (3) “Macromolecular crowding”: thermodynamic consequences for protein-protein interactions within the T4 DNA replication complex: The role of ATP hydrolysis”; (4) “Macromolecular crowding”: thermodynamic consequences for protein-protein interactions within the T4 DNA replication complex” at J Biol Chem. 1990 Sep. 5; 265(25):15160-7; (5) “Assembly of a functional replication complex without ATP hydrolysis: a direct interaction of bacteriophage T4 gp45 with T4 DNA polymerase” at Proc Natl Acad Sci USA. 1993 Apr. 15; 90(8):3211-5; and (6) “A coupled complex of T4 DNA replication helicase (gp41) and polymerase (gp43) can perform rapid and processive DNA strand-displacement synthesis” at Proc Natl Acad Sci USA. 1996 Dec. 10; 93(25):14456-61.

Target nucleic acids of the present invention include but are not limited to those nucleic acids associated with the development or onset of a disease state, including for example those nucleic acids that show the presence of specific infective agents or inherent defects of in an organism's genome. Target nucleic acids include but are not limited to nucleic acids that are exogenous and/or endogenous to the organism being screened. Exemplar target nucleic acids belonging to specific infective agents of interest include but are not limited to those nucleic acids derived from protozoa, parasites, fungi, bacteria, viruses, and combinations or derivatives thereof.

An object of the present invention is to provide methods and materials for the isothermal detection and screening of nucleic acids associated with breast cancer. In particular, the reagents are in the form of primer and probe sets which can be employed according to isothermal nucleic acid amplification procedures described herein to specifically and sensitively detect nucleic acids associated with breast cancer. Preferably, the primer/probe sets herein provided comprise two primer sequences and one probe sequence and are employed according to a reverse transcriptase (RT) cDNA sysnthesis step in combination with the strand transferase dependent isothermal amplification and detection format described herein. Primer/probe sets for the amplification and detection of nucleic acids associated with breast cancer have been described in U.S. Pat. No. 6,770,435, Reagents and methods useful for detecting diseases of the breast. This reference is herein incorporated by reference. U.S. Pat. No. 6,770,435 discloses a gene, or a fragment thereof, which codes for a BU101 polypeptide, and further discloses a BU101 gene, or a fragment thereof.

A set of contiguous and partially overlapping cDNA sequences and polypeptides encoded thereby, designated as BU101 and transcribed from breast tissue, is described in U.S. Pat. No. 6,770,435. These sequences are useful for the detecting, diagnosing, staging, monitoring, prognosticating, in vivo imaging, preventing or treating, or determining the predisposition of an individual to diseases and conditions of the breast, such as breast cancer. Also provided are antibodies which specifically bind to BU101-encoded polypeptide or protein, and agonists or inhibitors which prevent action of the tissue-specific BU101 polypeptide, which molecules are useful for the therapeutic treatment of breast diseases, tumors or metastases.

Certain aspects of the present invention provide methods and materials for detecting a target BU101 polynucleotide in a test sample which comprises contacting the test sample with at least one BU101-specific polynucleotide, a strand transferase, at least one polymerase, an amplification reaction mixture, and detecting the presence of an amplified target BU101 polynucleotide in the test sample. The BU101-specific polynucleotide has at least 50% identity with a polynucleotide selected from the group consisting of SEQUENCE ID NO 1, SEQUENCE ID NO 2, SEQUENCE ID NO 3, SEQUENCE ID NO 4, SEQUENCE ID NO 5, SEQUENCE ID NO 6, and fragments or complements thereof. Also, the BU101-specific polynucleotide may be attached to a solid phase prior to performing the method.

The present invention also provides a method for detecting BU101 mRNA in a test sample, which comprises performing reverse transcription (RT) with at least one primer in order to produce cDNA, amplifying the cDNA according to the isothermal DNA amplification technology disclosed herein using BU101 oligonucleotides as sense and antisense primers to obtain BU101 amplicon, and detecting the presence of the BU101 amplicon as an indication of the presence of BU101 mRNA in the test sample, wherein the BU101 oligonucleotides have at least 50% identity with a sequence selected from the group consisting of SEQUENCE ID NO 1, SEQUENCE ID NO 2, SEQUENCE ID NO 3, SEQUENCE ID NO 4, SEQUENCE ID NO 5, SEQUENCE ID NO 6, and fragments or complements thereof Amplification can be performed by the polymerase chain reaction. Also, the test sample can be reacted with a solid phase prior to performing the method, prior to amplification or prior to detection. This reaction can be a direct or an indirect reaction. Further, the detection step can comprise utilizing a detectable label capable of generating a measurable signal. The detectable label can be attached to a solid phase.

The present invention further provides a method of detecting a target BU101 polynucleotide in a test sample suspected of containing target BU101 polynucleotides, which comprises (a) contacting the test sample with at least one BU101 oligonucleotide as a sense primer and at least one BU101 oligonucleotide as an anti-sense primer, and amplifying according to the isothermal DNA amplification technology disclosed herein to obtain a first stage reaction product; (b) contacting the first stage reaction product with at least one other BU101 oligonucleotide to obtain a second stage reaction product, with the proviso that the other BU101 oligonucleotide is located 3′ to the BU101 oligonucleotides utilized in step (a) and is complementary to the first stage reaction product; and (c) detecting the second stage reaction product as an indication of the presence of a target BU101 polynucleotide in the test sample. The BU101 oligonucleotides selected as reagents in the method have at least 50% identity with a sequence selected from the group consisting of SEQUENCE ID NO 1, SEQUENCE ID NO 2, SEQUENCE ID NO 3, SEQUENCE ID NO 4, SEQUENCE ID NO 5, SEQUENCE ID NO 6, and fragments or complements thereof. Amplification may be performed by the polymerase chain reaction. The test sample can be reacted either directly or indirectly with a solid phase prior to performing the method, or prior to amplification, or prior to detection. The detection step also comprises utilizing a detectable label capable of generating a measurable signal; further, the detectable label can be attached to a solid phase.

Test kits useful for detecting target BU101 polynucleotide in a test sample are also provided which comprise a container containing at least one BU101 specific polynucleotide selected from the group consisting of SEQUENCE ID NO 1, SEQUENCE ID NO 2, SEQUENCE ID NO 3, SEQUENCE ID NO 4, SEQUENCE ID NO 5, SEQUENCE ID NO 6, and fragments or complements thereof. These test kits further comprise containers with tools useful for collecting test samples (such as, for example, blood, urine, saliva and stool). Such tools include lancets and absorbent paper or cloth for collecting and stabilizing blood; swabs for collecting and stabilizing saliva; and cups for collecting and stabilizing urine or stool samples. Collection materials, such as papers, cloths, swabs, cups, and the like, may optionally be treated to avoid denaturation or irreversible adsorption of the sample. The collection materials also may be treated with or contain preservatives, stabilizers or antimicrobial agents to help maintain the integrity of the specimens. the test kit will also include reagent for the isothermal amplication and detection of target nucleic acids associated with breast cancer. These include but are not limited to a reverse transcriptase, strand transferase, and a DNA dependent DNA polymerase.

The term “test sample” as used herein, means anything suspected of containing an BU101 target sequence, or other target nucleic acid associated with the development or onset of breast cancer. The test sample is, or can be derived from, any biological source, such as for example, blood, seminal fluid, ocular lens fluid, cerebral spinal fluid, milk, ascites fluid, synovial fluid, peritoneal fluid, amniotic fluid, tissue, fermentation broths, cell cultures and the like. The test sample can be used (i) directly as obtained from the source or (ii) following a pre-treatment to modify the character of the sample. Thus, the test sample can be pre-treated prior to use by, for example, preparing plasma from blood, disrupting cells or viral particles, preparing liquids from solid materials, diluting viscous fluids, filtering liquids, distilling liquids, concentrating liquids, inactivating interfering components, adding reagents, purifying nucleic acids, and the like.

A “target sequence” as used herein includes but is not limited to a nucleic acid sequence that is amplified, detected, or both amplified and detected using the primer sets herein provided. Additionally, while the term target sequence is sometimes referred to as single stranded, those skilled in the art will recognize that the target sequence may actually be double stranded. Thus, in cases where the target is double stranded, primer sequences of the present invention will amplify both strands of the target sequence.

The primer sets that can be employed to amplify a breast cancer target sequence preferably comprise deoxyribonucleic acid (DNA), or ribonucleic acid (RNA). Such primer sets can be employed according to isothermal DNA amplification disclosed herein. Additionally, in light of the presence cancer mRNA as an indicator for the onset of breast cancer, the isothermal nucleic acid amplification technology disclosed herein, and the primer sets may be employed in combination with a reverse transcriptase. Briefly, the reverse transcriptase provides a method of transcribing a strand of DNA from an RNA target sequence. The copied DNA strand transcribed from the RNA target is commonly referred to as “cDNA” which then can serve as a template for amplification by the isothermal nucleic acid amplification system mentioned above. The process of generating cDNA shares many of the hybridization and extension principles surrounding the isothermal nucleic acid amplification system described herein, but at least one of the enzymes employed should have reverse transcriptase activity. Enzymes having reverse transcriptase activity are well known and therefore don't warrant further discussion. Additionally, other methods for synthesizing cDNA are also known and include commonly owned U.S. patent application Ser. No. 08/356,287 filed Feb. 22, 1995, which is herein incorporated by reference. Generally, therefore, amplifying a breast cancer target sequence in a test sample will generally comprise the steps of contacting a test sample with a primer set, a strand transferase, a polymerase, and amplification reagents to form a reaction mixture and placing the reaction mixture under amplification conditions to thereby amplify the target sequence.

Amplification products produced using the primer sets provided herein may be detected using a variety of detection technologies well known in the art. For example, amplification products may be detected using agarose gel electrophoresis and visualization by ethidium bromide staining and exposure to Ultraviolet (UV) light or by sequence analysis of the amplification product for confirmation of breast cancer.

Alternatively, amplification products may be detected by oligonucleotide hybridization with a probe. Probe sequences generally are 10 to 50 nucleotides long, more typically 15 to 40 nucleotides long, and similarly to primer sequences, probe sequences are also nucleic acid. Hence, probes may comprise DNA, RNA or nucleic acid analogs such as uncharged nucleic acid analogs including but not limited to peptide nucleic acids (PNAs) which are disclosed in International Patent Application WO 92/20702 or morpholino analogs which are described in U.S. Pat. Nos. 5,185,444, 5,034,506, and 5,142,047 all of which are herein incorporated by reference. Such sequences can routinely be synthesized using a variety of techniques currently available. For example, a sequence of DNA can be synthesized using conventional nucleotide phosphoramidite chemistry and the instruments available from Applied Biosystems, Inc, (Foster City, Calif.); DuPont, (Wilmington, Del.); or Milligen, (Bedford, Mass.). Similarly, and when desirable, all nucleic acids disclosed herein, including but not limited to amplified target nucleic acids, primers, probes, or any combination thereof can be labeled using methodologies well known in the art such as described in U.S. Pat. Nos. 5,464,746; 5,424,414; and 4,948,882 all of which are herein incorporated by reference. Additionally, probes typically hybridize with the target sequence between the primer sequences. In other words, the probe sequence typically is not coextensive with either primer.

The term “label” as used herein means a molecule or moiety having a property or characteristic which is capable of detection. A label can be directly detectable, as with, for example, radioisotopes, fluorophores, chemiluminophores, enzymes, colloidal particles, fluorescent microparticles and the like; or a label may be indirectly detectable, as with, for example, specific binding members. It will be understood that directly detectable labels may require additional components such as, for example, substrates, triggering reagents, light, and the like to enable detection of the label. When indirectly detectable labels are used, they are typically used in combination with a “conjugate”. A conjugate is typically a specific binding member which has been attached or coupled to a directly detectable label. Coupling chemistries for synthesizing a conjugate are well known in the art and can include, for example, any chemical means and/or physical means that does not destroy the specific binding property of the specific binding member or the detectable property of the label. As used herein, “specific binding member” means a member of a binding pair, i.e., two different molecules where one of the molecules through, for example, chemical or physical means specifically binds to the other molecule. In addition to antigen and antibody specific binding pairs, other specific binding pairs include, but are not intended to be limited to, avidin and biotin; haptens and antibodies specific for haptens; complementary nucleotide sequences; enzyme cofactors or substrates and enzymes; and the like.

Probe sequences can be employed using a variety of homogeneous or heterogeneous methodologies to detect amplification products. Generally all such methods employ a step where the probe hybridizes to a strand of an amplification product to form an amplification product/probe hybrid. The hybrid can then be detected using labels on the amplified product, the primer, the probe or any combination thereof. Examples of homogeneous detection platforms for detecting amplification products include the use of FRET (fluorescence resonance energy transfer) labels attached to probes that emit a signal in the presence of the target sequence. So-called TaqMan assays described in U.S. Pat. No. 5,210,015 (herein incorporated by reference) and Molecular Beacon assays described in U.S. Pat. No. 5,925,517 (herein incorporated by reference) are examples of techniques that can be employed to homogeneously detect nucleic acid sequences. According to homogenous detection techniques, products of the amplification reaction can be detected as they are formed or in a so-called real time manner. As a result, amplification product/probe hybrids are formed and detected while the reaction mixture is under amplification conditions.

Heterogeneous detection formats typically employ a capture reagent to separate amplified sequences from other materials employed in the reaction. Capture reagents typically are a solid support material that is coated with one or more specific binding members specific for the same or different binding members. A “solid support material”, as used herein, refers to any material which is insoluble, or can be made insoluble by a subsequent reaction. Solid support materials thus can be a latex, plastic, derivatized plastic, magnetic or non-magnetic metal, glass or silicon surface or surfaces of test tubes, microtiter wells, sheets, beads, microparticles, chips, and other configurations known to those of ordinary skill in the art. To facilitate detection of an amplification product/probe hybrid in a heterogeneous type manner, primer or probes or both can be labeled with a first binding member which is specific for its binding partner which is attached to a solid support material such as a microparticle. Similarly, primers may be labeled with a second binding member specific for a conjugate as defined above. The amplification products bound to the probes can then be separated from the remaining reaction mixture by contacting the reaction mixture with the above solid support and then removing the solid support from the reaction mixture. Any amplification product/probe hybrids bound to the solid support may then be contacted with a conjugate to detect the presence of the hybrids on the solid support.

Whether detected in a homogeneous or heterogeneous manner, methods for detecting a target sequence in a test sample will generally comprise the steps of contacting a test sample with a primer set provided herein, a strand transferase, a polymerase, and amplification reagents to form a reaction mixture. The reaction mixture then is placed under amplification conditions to form an amplification product, as specified above. The amplification product is then detected as an indication of the presence of the target sequence in the test sample. As stated above, the reaction product may be detected using gel electrophoresis, heterogeneous methods or homogeneous methods. Accordingly, the reaction product may be detected in the reaction mixture while it is under amplification conditions with homogeneous techniques. Alternatively, the amplification product may be detected after amplification of the target sequence is complete using heterogeneous techniques or gels.

The present invention also provides oligonucleotide sets useful for amplifying and detecting breast cancer target sequence in a test sample. These oligonucleotide sets, or “oligo sets”, comprise a primer set and a molecular beacon probe that can be used in the manner set forth above. Additionally, the oligo sets may be packaged in suitable containers and provided with additional reagents such as, for example, amplification reagents (also in suitable containers) to provide kits for detecting breast cancer target nucleic acids in a test sample.

In one embodiment of the present invention, a test sample is reacted with a primer set either alone or in combination with other primer sets in a multiplex reaction. The test sample and the primers are reacted in combination with a strand transferase, a polymerase, and amplification reagents to produce an amplified target nucleic acid that can be detected either by detection of a labeled probe, or by detection of a labeled incorporated nucleotide, or by detection of a labeled primer, or by a combination thereof.

In another embodiment of the present invention, a test sample is reacted with a single primer, or with two or more single primers selected from different primer sets in a multiplex reaction, whereby said primer or primers are complementary to the same strand of nucleic acid. The test sample and the primers can be reacted in combination with a strand transferase, a polymerase, and amplification reagents to produce amplified single stranded target nucleic acid that can be detected either by the detection of a labeled probe, or by the detection of an labeled incorporated nucleotide, or by the detection of a labeled primer, or by a combination thereof.

In certain embodiments of the present invention, a target nucleic acid is detected by reacting a test sample and any one or more primers or primer sets with a reverse transcriptase polymerase to produce a first cDNA. Reaction of the test sample and the primer or primers with the reverse transcriptase to produce a cDNA is performed either before or concomitant with the admixture of a strand transferase, a polymerase, and amplification reagents. The amplified target nucleic acid can be detected either by the detection of a labeled probe, or by the detection of an labeled incorporated nucleotide, or by the detection of a labeled primer, or by a combination thereof.

While the invention has been described in detail and with reference to specific embodiments, it will be apparent to one skilled in the art that various changes and modifications may be made to such embodiments without departing from the spirit and scope of the invention.

What is claimed is:

1. A method for detecting the presence of a breast cancer target nucleic acid in a test sample comprising contacting a test sample with a strand transferase, a polymerase, and at least one primer having complementarity to said breast cancer target nucleic acid.

2. The method of claim 1 wherein said strand transferase is derived from a prokaryotic.

3. The method of claim 1 wherein said strand transferase is the uvsX strand transferase derived from the bacteriophage T4.

4. The polymerase of claim 1 wherein said polymerase is derived from a prokaryotic.

5. The polymerase of claim 1 wherein said polymerase is the gp43 polymerase derived from the bacteriophage T4.


SEQUENCE LISTING (19 sequences)

SEQ ID NO: 1 - DNA, 274 bp, Homo Sapiens
aaatagccctgggctctgcagctccacaggctcctggggtggagtccaaatcactcattgtttgtgaaagctgagctcacagcaaaacaagccaccatgaagctgtcggtgtgtctcctgctggtcacgctggccctctgctgctaccaggccaatgccgagttctgcccagctcttgtttctgagctgttagacttcttcttcattagtgaacctctgttcaagttaagtcttgccaaatttgatgcccctctggaagctgttgcagccaagt

SEQ ID NO: 2 - DNA, 237 bp, Homo Sapiens
tccaaatcactcattgtttgtgaaagctgagctcacagcaaaacaagccaccatgaagctgtcggtgtgtctcctgctggtcacgctggccctctgctgctaccaggccaatgccgagttctgcccagctcttgtttctgagctgttagacttcttcttcattagtgaacctctgttcaagttaagtcttgccaaatttgatgcccctccggaagctgttgcagccaagttaggagt

SEQ ID NO: 3 - DNA, 230 bp, Homo Sapiens
ccggaagctgttgcagccaagttaggagtgaagagatgcacggatcagatgtcccttcagaaacgaagcctcattgcggaagtcctggtgaaaatattgaagaaatgtagtgtgtgacatgtaaaaactttcatcctggtttccactgtctttcaatgacaccctgatcttcactgcagaatgtaaaggtttcaacgtcttgctttaataaatcacttgctctccacgtc

SEQ ID NO: 4 - DNA, 478 bp, Homo Sapiens
aaatagccctgggctctgcagctccacaggctcctggggtggagtccaaatcactcattgtttgtgaaagctgagctcacagcaaaacaagccaccatgaagctgtcggtgtgtctcctgctggtcacgctggccctctgctgctaccaggccaatgccgagttctgcccagctcttgtttctgagctgttagacttcttcttcattagtgaacctctgttcaagttaagtcttgccaaatttgatgcccctctggaagctgttgcagccaagttaggagtgaagagatgcacggatcagatgtcccttcagaaacgaagcctcattgcggaagtcctggtgaaaatattgaagaaatgtagtgtgtgacatgtaaaaactttcatcctggtttccactgtctttcaatgacaccctgatcttcactgcagaatgtaaaggtttcaacgtcttgctttaataaatcacttgctctcca

SEQ ID NO: 5 - DNA, 438 bp, Homo Sapiens
gtccaaatcactcattgtttgtgaaagctgagctcacagcaaaacaagccaccatgaagctgtcggtgtgtctcctgctggtcacgctggccctctgctgctaccaggccaatgccgagttctgcccagctcttgtttctgagctgttagacttcttcttcattagtgaacctctgttcaagttaagtcttgccaaatttgatgcccctccggaagctgttgcagccaagttaggagtgaagagatgcacggatcagatgtcccttcagaaacgaagcctcattgcggaagtcctggtgaaaatattgaagaaatgtagtgtgtgacatgtaaaaactttcatcctggtttccactgtctttcaatgacaccctgatcttcactgcagaatgtaaaggtttcaacgtcttgctttaataaatcacttgctctccacgt

SEQ ID NO: 6 - DNA, 481 bp, Homo Sapiens
aaatagccctgggctctgcagctccacaggctcctggggtggagtccaaatcactcattgtttgtgaaagctgagctcacagcaaaacaagccaccatgaagctgtcggtgtgtctcctgctggtcacgctggccctctgctgctaccaggccaatgccgagttctgcccagctcttgtttctgagctgttagacttcttcttcattagtgaacctctgttcaagttaagtcttgccaaatttgatgcccctccggaagctgttgcagccaagttaggagtgaagagatgcacggatcagatgtcccttcagaaacgaagcctcattgcggaagtcctggtgaaaatattgaagaaatgtagtgtgtgacatgtaaaaactttcatcctggtttccactgtctttcaatgacaccctgatcttcactgcagaatgtaaaggtttcaacgtcttgctttaataaatcacttgctctccacgt

SEQ ID NO: 7 - DNA, 68 bp, Artificial Sequence
  Modified Polylinker Fragments
agctcggaattccgagcttggatcctctagagcggccgccgactagtgagctcgtcgacccgggaatt

SEQ ID NO: 8 - DNA, 68 bp, Artificial Sequence
  Modified Polylinker Fragments
aattaattcccgggtcgacgagctcactagtcggcggccgctctagaggatccaagctcggaattccg

SEQ ID NO: 9 - DNA, 32 bp, Artificial Sequence
  Universal Primer
gaattccgagcggataacaatttcacacagga

SEQ ID NO: 10 - DNA, 18 bp, Artificial Sequence
  Universal Primer
tgtaaaacgacggccagt

SEQ ID NO: 11 - DNA, 19 bp, Artificial Sequence
  Primer
acttgaacagaggttcact

SEQ ID NO: 12 - DNA, 20 bp, Artificial Sequence
  Primer
cagccaagttaggagttgaa

SEQ ID NO: 13 - DNA, 19 bp, Artificial Sequence
  Sense Primer
gagatgcacggatcagatg

SEQ ID NO: 14 - DNA, 20 bp, Artificial Sequence
  Antisense Primer
ttttacgtggagagcaagtg

SEQ ID NO: 15 - DNA, 24 bp, Artificial Sequence
  Forward Primer
ttctgcccagctcttgtttctgag

SEQ ID NO: 16 - DNA, 19 bp, Artificial Sequence
  Reverse Primer
tgcagtgaagatcagggtg

SEQ ID NO: 17 - DNA, 13 bp, Artificial Sequence
  Probe
gcctcattgcgga

SEQ ID NO: 18 - DNA, 20 bp, Artificial Sequence
  Sense Primer
taatacgactcactataggg

SEQ ID NO: 19 - DNA, 30 bp, Artificial Sequence
  Antisense Primer
gcggccgcccacactacatttcttcaatat